(19) United States
(12) Patent Application Publication
(10) Pub. No.: US 2005/0202461 A1
(43) Pub. Date: Sep. 15, 2005

(54) Method for converting generic nucleic acid priming sequences

(76) Inventors: Robert C. Getts, Collegeville, PA (US); Jammes Kadushin, Gilbertsville, PA (US); Jaime Schwalm, CollegeTown, PA (US); Kevin Howerton, Pipersville, PA (US)

Correspondence Address: LAW OFFICE OF MORRIS E. COHEN, 1122 CONEY ISLAND AVENUE, SUITE 217, BROOKLYN, NY, 11230, US

(21) Appl. No.: 10/951,549

(22) Filed: Sep. 27, 2004

Related U.S. Application Data
(63) Continuation of application No. PCT/US03/09232, filed on Mar. 25, 2003.
(63) Continuation-in-part of application No. 10/825,776, filed on Apr. 16, 2004.
Application No. 10/825,776 is a continuation of application No. 10/050,088, filed on Jan. 14, 2002, now abandoned.
(63) Continuation-in-part of application No. 10/730,823, filed on Dec. 8, 2003.
Application No. 10/730,823 is a continuation of application No. 10/393,519, filed on Mar. 20, 2003, now abandoned.
Application No. 10/393,519 is a continuation of application No. PCT/US01/29589, filed on Sep. 20, 2001.
Application No. 10/393,519 is a continuation-in-part of application No. 09/908,950, filed on Jul. 19, 2001.
(63) Continuation-in-part of application No. 09/908,950, filed on Jul. 19, 2001.
(63) Continuation-in-part of application No. 09/802,162, filed on Mar. 8, 2001.
(60) Provisional application No. 60/367,438, filed on Mar. 25, 2002.
(60) Provisional application No. 60/261,231, filed on Jan. 13, 2001.
(60) Provisional application No. 60/234,060, filed on Sep. 20, 2000.
(60) Provisional application No. 60/219,397, filed on Jul. 19, 2000.
(60) Provisional application No. 60/187,681, filed on Mar. 8, 2000.

(51) Int. Cl. C12Q 1/68

(52) U.S. Cl. 435/6

(57) ABSTRACT

Methods for assay and/or amplification of nucleic acid sequences. An amplification procedure is provided for use with RNA samples that do not have a poly A sequence on the 3 prime end of the messenger RNA as is the case for bacterial, and other total RNA sources. Further, a method is provided for coupling, via ligation, a nucleic acid sequence to the 5 prime end of a random or sequence specific primer or to the 3 prime or 5 prime end of a synthesized DNA probe or target sequence, preferably to enable labelling of the target sequence or amplification thereof.

### RELATED APPLICATIONS

The present application is a continuation of PCT Application Serial No. PCT/US03/09232 filed 25 Mar. 2003 (pending) (“the '232 application”) which claims the priority of U.S. Provisional Application Ser. No. 60/367,438 filed Mar. 25, 2002.

The present application is also a continuation-in-part of U.S. Nonprovisional application Ser. No. 10/825,776 filed Apr. 16, 2004 (pending), which is a continuation of U.S. Nonprovisional application Ser. No. 10/050,088 filed Jan. 14, 2002 (abandoned), which claims the priority of U.S. Provisional Application Ser. No. 60/261,231 filed Jan. 13, 2001.

The present application is also a continuation-in-part of U.S. Nonprovisional application Ser. No. 10/730,823 filed Dec. 8, 2003 (pending) which is a continuation of U.S. Nonprovisional application Ser. No. 10/393,519 filed Mar. 20, 2003 (abandoned), which is a continuation of PCT Application Serial No. PCT/US01/29589 filed Sep. 20, 2001 (abandoned), which claims the priority of U.S. Provisional Application Ser. No. 60/234,060 filed Sep. 20, 2000. PCT Application Serial No. PCT/US01/29589 filed Sep. 20, 2001 is a continuation-in-part of U.S. Nonprovisional application Ser. No. 09/908,950 filed Jul. 19, 2001 (pending), and also claims the priority of U.S. Provisional Application Ser. No. 60/219,397, filed Jul. 19, 2000, and the priority of U.S. Provisional Application Ser. No. 60/187,681 filed Mar. 8, 2000.

The present application is also a continuation-in-part of U.S. Nonprovisional application Ser. No. 09/908,950 filed Jul. 19, 2001 (pending), which claims the priority of U.S. Provisional Application Ser. No. 60/219,397, filed Jul. 19, 2000.

The present application is also a continuation-in-part of U.S. Nonprovisional application Ser. No. 09/802,162 filed Mar. 8, 2001 (pending), which claims the priority of U.S. Provisional Application Ser. No. 60/187,681 filed Mar. 8, 2000.

The priority of all of those prior applications is claimed herein, all of which are fully incorporated herein by reference.

### FIELD OF THE INVENTION

The present invention is related generally to nucleic acid assays, more particularly to methods of hybridization in solution or on surfaces, like microarrays, and methods of amplification, like linear amplification using RNA polymerase.

### BACKGROUND OF THE INVENTION

Nucleic acid detection is traditionally performed by hybridizing two complementary strands of nucleic acid (DNA or RNA), one of which is the target and one of which is the probe, labeled nucleotides having been incorporated directly into one of the two strands to generate a detectable signal. The label may be a radioisotope such as ³²P, biotin, digoxigenin, various fluorescent molecules, or so forth, as is well known in the art. Alternatively, the label may be added indirectly by first incorporating either a functional moiety, like in the CyScribe Post labeling kit (Amersham) which can be chemically coupled to a label in a secondary reaction. In a second indirect approach, like the Submicro labeling kit (Genisphere Inc), a defined nucleic acid sequence can be included in probe or target synthesis. This sequence can capture a labeled DNA dendrimer. In a typical assay, one of the two complementary nucleic acid strands is usually attached to some type of a support, such as a membrane (as with Southern and northern blots), or such as a glass slide (as with microarrays). Usually a solid support is used, although other types of supports have also been disclosed in the art.

Generally, a microarray comprises a substantially planar substrate such as a glass microscope slide, a silicon plate or nylon membrane, coated with a grid of tiny spots or features of about 20 microns in diameter. Each spot or feature contains millions of copies of a specific sequence of nucleic acid extracted from a strand of deoxyribonucleic acid (DNA). Each microarray is capable of performing the equivalent of thousands of individual “test tube” experiments over a short time period thereby providing rapid and simultaneous detection of thousands of genes. Microarrays have been implemented in a range of applications such as analyzing a sample for the presence of gene variations or mutations (i.e. genotyping), or for gene expression profiling.

In a gene expression analysis, messenger RNA (mRNA) is extracted from a sample of cells. The mRNA, serving as a template, is reverse transcribed to yield a complementary DNA (cDNA). As a first example of the prior art techniques, one or more labels or markers such as fluorescent dyes are directly incorporated into the copies of cDNA during the reverse transcription process. Under suitable hybridization conditions, the labeled fragments are hybridized or coupled with complementary nucleic acid sequences (i.e. gene probes) attached to the features of the microarray for ready detection thereof. This labeling method has been commonly referred to as “direct incorporation”.

Upon hybridization of the cDNA to the microarray, a detectable signal (e.g. fluorescence) is emitted for a positive outcome from each feature containing a cDNA fragment hybridized with a complementary gene probe attached thereto. The detectable signal is visible to an appropriate sensor device or microscope, and may then be analyzed by the computer or user to generate a hybridization pattern. Since the nucleic acid sequence at each feature on the array (the probe) is known, any positive outcome (i.e. signal generation) at a particular feature indicates the presence of the complementary cDNA sequence in the sample cell. Although there are occasional mismatches, the attachment of millions of gene probes at each spot or feature ensures that the detectable signal is strongly emitted only if the complementary cDNA of the test sample is present.

As a second example of the prior art techniques, one or more labels or markers such as fluorescent dyes are indirectly incorporated into the copies of cDNA by attachment to a capture sequence after the reverse transcription process. This labeling method has been configured as a commercially available detection kit (Genisphere, Inc., Montvale N.J.), referred to as “Submicro Expression Array Detection Kit” and involves the detection (indirectly) of a capture sequence attached to the primer used during the reverse transcription by a fluorescent labeled DNA dendrimer. Complementary DNA is prepared from the RNA sample (e.g. total RNA or poly(A)+ RNA) through conventional techniques for implementing reverse transcription using reverse transcription primers (RT primers) having a defined sequences of nucleotides, referred to as a “capture” sequence, attached to the 5 prime end of a short poly dT sequence. This results in the formation of the target cDNA with the capture sequence located at the 5′ end. The newly formed target cDNA with the capture sequence is then isolated from the mRNA sample and hybridized to the complementary gene probes affixed to the microarray. After the target cDNA and the microarray are hybridized, the microarray is washed to remove any excess RT primers prior to labeling. A mixture containing labeled “dendritic nucleic acid molecules”, or “dendrimers”, is then prepared. The mixture is formulated in the presence of a suitable buffer to yield a dendrimer hybridization mixture containing dendrimers with labels attached to any or all of the arms, and with oligonucleotides complementary to the capture sequences of the target cDNA attached to one or more of the arms. The labeled dendrimers are added to the microarray for hybridization of the capture sequence complement of the dendrimer with the capture sequences of the bound cDNA probe to generate a detectable signal from the corresponding feature. The microarray is washed to remove any excess unhybridized dendrimer molecules to reduce unwanted noise generation. The microarray is scanned using conventional techniques to detect the signal emitted by the labels to generate a particular hybridization pattern for analysis. Detected signal indicates the presence of labeled dendrimers hybridized to cDNA bound to a feature (a probe) on the microarray. Since the probes affixed to the each position on the microarray are of known sequence, the signal provides important sequence information about the previously unknown sequences of the sample.

Dendrimers are complex, highly branched molecules, and are comprised of a plurality of interconnected natural or synthetic monomeric subunits of double-stranded DNA forming stable spherical-like or dendritic core structures with a predetermined number of “free ends” or “arms” extending therefrom. Dendrimers provide efficient means for labeling reactions such as fluorescence, for example, and facilitate direct calculations of the number of transcripts bound due to their predetermined signal generation intensity and proportional relationship to the bound cDNA on the microarray.

Each dendrimer includes two types of hybridization “free ends” or “arms” extending from the core surface. Each dendrimer may be configured to include at least one hundred arms of each type. The arms are composed of a single-stranded DNA of a specific sequence that can be ligated or hybridized to a functional molecule, such as a target molecule or a label. The dendrimer in conjunction with the target molecule has the capability to target and hybridize to a complementary sequence of probe affixed to the array. The label molecule can be attached to the other type of arm to provide the dendrimer with signal emission capabilities for detection of the dendrimer, signaling a hybridization event thereof. The dendrimer is typically hybridized to the target molecule by providing a nucleotide sequence on an arm that is complementary to the capture sequence of the target molecule, and the label molecule is typically an oligonucleotide linked to a label or marker which is attached to a dendrimer. Using simple DNA labeling, hybridization, and ligation reactions, a dendrimer can thus be configured to act as a highly labeled, target-specific molecule, and therefore may be used in a microarray system for DNA analysis. Dendrimer technology is described in greater detail in U.S. Pat. Nos. 5,175,270 and 5,484,904, in Nilsen et al., Dendritic Nucleic Acid Structures, J. Theor. Biol., 187, 273-284 (1997); in Stears et al., A Novel, Sensitive Detection System for High-Density Microarrays Using Dendrimer Technology, Physiol. Genomics, 3: 93-99 (2000); various applications of the present inventor, such as PCT Application Serial No. PCT/US01/07477; and published protocols available from Genisphere, Inc. of Montvale, N.J., all of which are fully incorporated herein by reference.

It would be desirable to have an indirect probe labeling method using dendrimer technology that would be adaptable to RNA samples that do not have 3 prime poly A sequences on the messenger RNA as is the case in bacterial and certain other RNA samples.

Further, as nucleic acid hybridization and detection technologies progress, it has become increasingly desirable to further improve the sensitivity of emerging standard detection assays, like those performed on microarrays. One example of such a method is the method of Eberwine commonly referred to as T7 Amplification (as discussed for example in U.S. Pat. Nos. 5,716,785; 5,891,636; 5,958,688; and 6,291,170 B1; all of which are fully incorporated herein by reference). Typically in this amplification method, complementary DNA is prepared from the RNA sample (e.g. total RNA or poly(A)+ RNA) through conventional techniques for implementing reverse transcription using modified, complexed reverse transcription primers having an RNA Polymerase promotor sequence (T7, SP6, T3, or the like RNA polymerase) attached 5 prime to a poly dT sequence. RNA samples must contain messenger RNA that contains a poly A sequence on the 3 prime end for successful reverse transcription. This results in the formation of the target cDNA with the RNA polymerase promoter located at the 5′ end. The newly formed single stranded cDNA with the promoter sequence is then converted into double stranded DNA using methods familiar to one experienced in the art. The double strand DNA is purified and cRNA generated from the second DNA strand using RNA polymerase and nucleotide triphosphates in the presence of a buffer suitable for RNA synthesis. Often the cRNA is referred to as amplified RNA (aRNA) because during the process of copying the second strand DNA into RNA the amount of RNA produced represents a linear increase (50-100 fold more copies) of the starting DNA. Kits are commercially available from multiple sources to conduct this amplification procedure (Ambion, Epicentre, etc.). Commonly, during the amplification procedure the aRNA is labeled with either a fluorescent dye nucleotide, a radionucleotide, a biotinylated nucleotide, or some other suitably labeled nucleotide. In one type of assay using such a labeled RNA molecule, a microarray, the labeled aRNA is fragmented and hybridized to the features on the microarray in a buffer suitable to promote hybridization of the aRNA to the probes on the surface. The microarray is scanned using conventional techniques to detect the signal emitted by the labels to generate a particular hybridization pattern for analysis. Signal detection indicates the presence of hybridization of molecules in the sample to a feature (a probe) on the microarray. Since the probes affixed to the each position on the microarray are of known sequence, the signal provides important sequence information about the previously unknown sequences of the sample.

Unfortunately, this amplification methodology is restricted to messenger RNA samples having 3 prime poly A sequences and requires a primer with greater than 20 additional nucleotides comprising the promoter sequence, extending from the 5 prime end of the reverse transcription primer. Longer primers often require more cumbersome puification procedures to remove excess material and may introduce unforeseen bias into the process. It would be desirable for a RNA polymerase amplification method to both work with samples containing messenger RNA that does not have a 3 prime poly A sequence (like prokaryotic or bacterial samples) and to reverse transcribe with a shorter primer.

### SUMMARY OF THE INVENTION

It is an object of the present invention to provide a method for assaying nucleic acid sequences.

It is an object of the present invention to provide a method for amplification of RNA.

It is a further object of the present invention to provide an amplification procedure for use with RNA samples that do not have a poly A sequence on the 3 prime end of the messenger RNA as is the case for bacterial, and other total RNA sources.

It is a further object of the present invention to provide a method for coupling, via ligation, a nucleic acid sequence to the 5 prime end of a random or sequence specific primer or to the 3 prime or 5 prime end of a synthesized DNA probe or target sequence. This sequence may serve to enable labelling of the target sequence, amplification thereof, or another desired purpose. For example this sequence may be designed to “capture” a label-containing molecule. In a preferred embodiment, that label-containing molecule (also referred to herein as a “signal molecule”), is a DNA dendrimer for labelling of the target sequence. Or, this sequence may serve as a promoter for the generation of amplified RNA from RNA samples without poly A sequences on the 3 prime end, as is the case for bacterial and some other total RNA sources.

It is a further object of the present invention to provide a method for linear amplification of starting RNA samples that does not require a reverse transcription primer having an RNA polymerase promoter with a poly dT on the 3 prime end. Rather, in one example of a preferred embodiment, the method couples the promoter to the end of a simple primer, like a random 9mer with a short defined sequence extension or a short oligo dT sequence (e.g. 17-24 bases), after the reverse transcription and prior to the amplification process.

It is a further object of the present invention to provide a method for linear amplification of starting DNA samples like genomic DNA or specifically primed DNA sequences.

Further objects and advantages of the invention will become apparent in conjunction with the detailed disclosure provided herein.

### BRIEF DESCRIPTION OF THE DRAWINGS

The following drawings are illustrative of embodiments of the invention and are not to be construed as limiting the invention as encompassed by the claims forming part of the application.

FIG. 1 is a diagramatic representation of the use of a bridging oligonucleotide, in accordance with the present invention.

FIG. 2 is a illustration of one example of a first embodiment of the present invention, using ligation followed by use of a capture sequence.

FIG. 3 is an illustration of one example of a further embodiment of the present invention, using ligation followed by linear amplification.

### DESCRIPTION OF THE INVENTION

In accordance with one embodiment of the present invention, the methods described may be applied to any assay type in which a probe or target is synthesized and hybridized to a complementary nucleic acid or recognition molecule such as a standard blot, microarray, microtiter plate, captive bead, flow-based format, in situ hybridization, etc.

Although reference will generally be made to microarrays for illustration purposes, it is to be understood that the invention is not limited to arrays, but may be used with blots or any other assay formats currently in use or later developed in the art. Similarly, while the use of DNA dendrimers as the label containing molecule constitutes a preferred embodiment, other labeling methodologies or capture reagents currently in use or later developed can be used as well, consistent with the invention. For example, the present invention can be used with direct incorporation of a labeled nucleotide into the generated probes or targets prior to use in a standard assay. In addition, molecules other than dendrimers can be “captured” to generate a signal. For example, a partially double stranded DNA molecule can be captured and then can generate a signal by detection using an anti-double-stranded antibody.

In one preferred embodiment of the invention, the first step of this invention involves the use of an oligonucleotide primer which is synthesized or obtained from a suitable source. This oligonucleotide primer has seven bases of defined sequence on its 5 prime end. These seven nucleotides of known sequence can either be part of a well known primer, like poly dT₁₇ in reverse transcription, or can be added to the end of the primer during synthesis. In the case of the former approach of a sequence defined primer, it is necessary to add a phosphate to the 5 prime end during synthesis. The seven 5 prime “T” nucleotides serve as the defined sequence and will serve as the bridging sequence as described later. An example of the latter approach would be a primer configured from 5 prime “PO₄-GCTTTTTNNNNNNNNN” 3 prime, where “PO₄” designates phosphorylation of the synthesized oligonucleotide after synthesis and “N” represents a random mixture of any of the 4 nucleotides that comprise DNA. The final synthesis pool of this primer will have oligonucleotides representing all of the possible combinations of the 4 nucleotides at the positions designated by “N”. The purpose of these seven 5 prime nucleotides, “GCTTTTT”, is to serve as a bridging point between the primer and a second oligonucleotide to be coupled to the end as described below.

The next step in the method is to use the prepared primer to generate a single stranded copy of DNA (cDNA), from a nucleic acid sequence of either RNA or DNA by the process of reverse transcription or DNA replication, respectively, using methods familiar to one of ordinary skill in the art. The excess primer is removed from the synthesized copy of DNA by standard methods known in the art, such as chromatography. The purified cDNA is then coupled to a second defined oligonucleotide sequence by ligation using DNA ligase in a buffer suitable for the ligase to function to couple the two oligonucleotide sequences. The second oligonucleotide sequence may be designed to “capture” a label-containing molecule like a DNA dendrimer, may serve as a promoter for the generation of amplified RNA, or may function as both capture sequence and RNA polymerase promoter, or may serve another desired purpose.

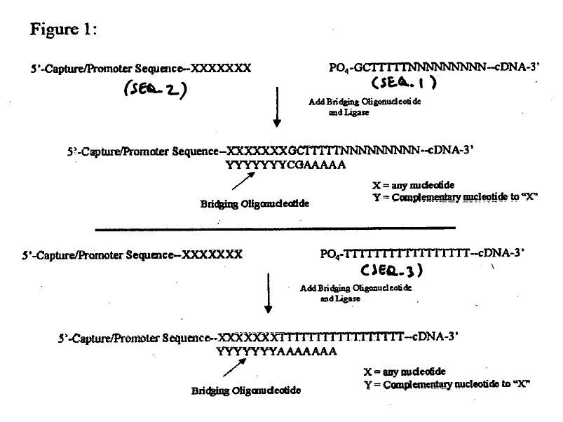

An additional requirement of the ligation process is use of a bridging oligonucleotide sequence whose function is to align the primer to the capture or promoter sequence. The bridging oligonucleotide is designed to be complementary to bases of the primer and to bases of the capture sequence. In the preferred embodiment, the bridging oligonucleotide is complementary to at least 7 bases of the primer and 7 bases of the capture or promoter sequence as illustrated in FIG. 1. Alternatively, hybridization can be conducted to fewer bases of primer and/or to fewer bases of the capture or promoter sequence.

In the preferred embodiment, the bridging oligonucleotide is added to the capture or promoter sequence in a prehybridization mixture prior to adding the combination of both to the cDNA for ligation using DNA ligase. Alternatively, if RNA ligase is used instead of DNA ligase, a bridging oligonucleotide is not necessary and the cDNA can be directly coupled to the capture or promoter sequence oligonucleotide. The capture sequence or promoter can be coupled to either the 3 prime or 5 prime end of the cDNA using either DNA or RNA ligase. Furthermore, RNA ligase can be used to couple the RNA to RNA, RNA to DNA, or DNA to DNA. It is to be understood that this invention should not be restricted to any one of these nucleic acid combinations.

Once the cDNA has been ligated to either the capture sequence or promoter and the ligation reaction has been terminated, the coupled product is taken into an assay specifically designed to operate based on the ligated sequence.

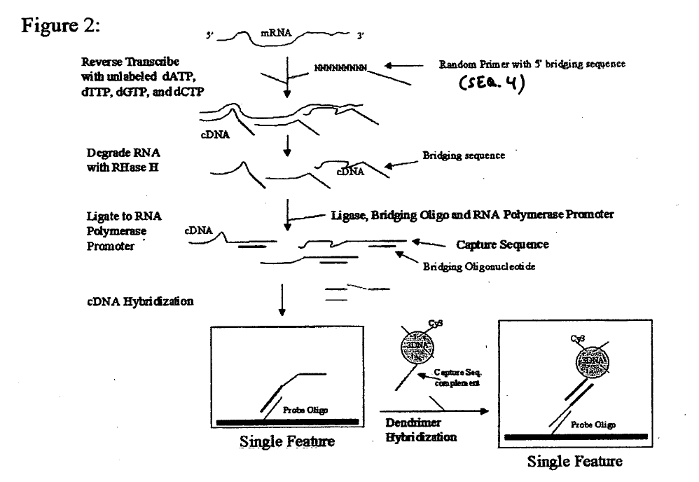

In one preferred embodiment of the invention, as shown with reference to FIG. 2, the 5 prime end of a random primed cDNA produced from a bacterial total RNA sample is ligated to a capture sequence using DNA ligase and the appropriate bridging oligonucleotide. The ligated cDNA/capture sequence mixture is applied to a microarray in a hybridization buffer suitable to allow binding of the cDNA to the probes on the array. The array is washed by methods familiar to one educated in the art. A fluorescent labeled DNA dendrimer with a sequence complementary to the ligated “capture” sequence of the cDNA is added to the array in a second hybridization step and in a suitable hybridization buffer to facilitate binding of the dendrimer to the capture sequence. The excess dendrimer is washed from the array and the label detected on a suitable fluoresence detection scanner.

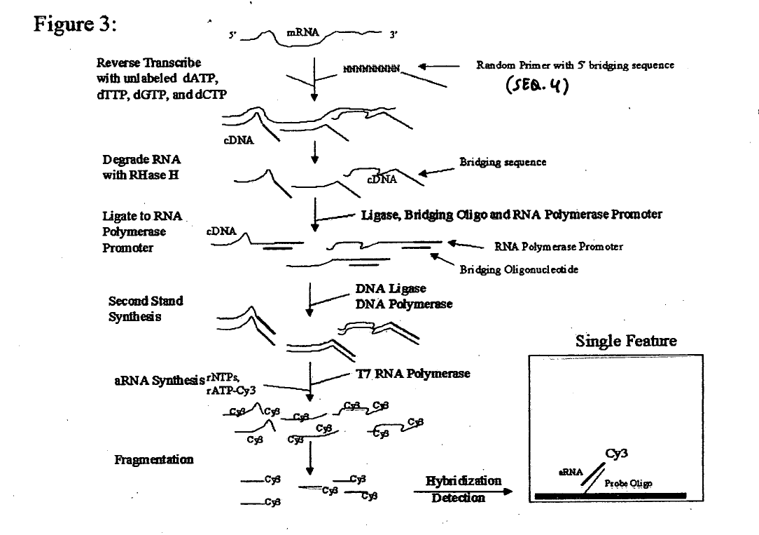

In another embodiment of the present invention, as shown with reference to FIG. 3, the 5 prime end of a random primed cDNA produced from a prokaryotic (bacterial) total RNA sample is ligated to an RNA polymerase promoter using DNA ligase and the appropriate bridging oligonucleotide. The ligated cDNA/promoter sequence is converted into double stranded DNA by methods familiar in the art. The mixture is purified to remove contaminating components and aRNA is generated using RNA polymerase and other required components in a suitable buffer. This can be accomplished using commercially available kits (Ambion and Epicentre). During the aRNA synthesis a fluorescent nucleotide can be directly incorporated into the aRNA. The aRNA is then fragmented by standard methods and is applied to a microarray in a buffer suitable to promote the hybridization of the labeled, fragmented aRNA to the probes on the microarray. The excess unbound aRNA is washed from the array and the label detected on a suitable fluoresence detection scanner.

In a further embodiment of the present invention, the 5 prime end of a cDNA produced from a mammalian total RNA sample using an oligo dT primer is ligated to an RNA polymerase promoter using DNA ligase and the appropriate bridging oligonucleotide. The ligated cDNA/promoter sequence is converted into double stranded DNA by methods familiar to one educated in the art. The mixture is purified to remove contaminating components and aRNA is generated using RNA polymerase and other required components in a suitable buffer. This can be accomplished using commercially available kits (Ambion and Epicentre). During the aRNA synthesis a fluorescent nucleotide can be directly incorporated into the aRNA. The aRNA is then fragmented by standard methods and is applied to a microarray in a buffer suitable to promote the hybridization of the labeled, fragmented aRNA to the probes on the microarray. The excess unbound aRNA is washed from the array and the label detected on a suitable fluoresence detection scanner.

In a still further embodiment of the present invention, the 5 prime end of a random primed cDNA produced from a bacterial total RNA sample is ligated to an oligonucleotide with RNA polymerase promoter and a dendrimer capture sequence using DNA ligase and the appropriate bridging oligonucleotide. The ligated cDNA/promoter capture sequence is converted into double stranded DNA by methods familiar to one educated in the art. The mixture is purified to remove contaminating components and aRNA is generated using RNA polymerase and other required components in a suitable buffer. This can be accomplished using commercially available kits (Ambion and Epicentre). The mixture is applied to a microarray in a buffer suitable to promote the hybridization of the labeled aRNA to the probes on the microarray. The excess unbound aRNA is washed from the array. A fluorescent labeled DNA dendrimer with a sequence complementary to the “capture” sequence of the aRNA is added to the array in a second hybridization step and in a suitable hybridization buffer to facilitate binding of the dendrimer to the capture sequence. The excess dendrimer is washed from the array and the label detected on a suitable fluoresence detection scanner.

In a still further embodiment of the present invention, the 5 prime end of a random primed cDNA produced from a genomic DNA sample is ligated to a capture sequence using DNA ligase and the appropriate bridging oligonucleotide. The ligated cDNA/capture sequence mixture is applied to a microarray in a hybridization buffer suitable to allow binding of the cDNA to the probes on the array. The array is washed by methods familiar to one educated in the art. A fluorescent labeled DNA dendrimer with a sequence complementary to the ligated “capture” sequence of the cDNA is added to the array in a second hybridization step and in a suitable hybridization buffer to facilitate binding of the dendrimer to the capture sequence. The excess dendrimer is washed from the array and the label detected on a suitable fluoresence detection scanner.

In still a further embodiment of the invention, the 5 prime end of a random primed cDNA produced from a restriction endonuclease digested genomic DNA sample is ligated to a RNA polymerase promoter using DNA ligase and the appropriate bridging oligonucleotide. The promoter is designed to include several additional nucleotides on the 5 prime end that contain phosphothiolate linkages between the nucleotides. This alteration will prevent the digestion of the primer or cDNA coupled to the 3 prime end of the primer by Exo5 which digests DNA from the 5 prime end. The original genomic DNA is degraded by Exo 5 and the ligated cDNA/promoter sequence is converted into double stranded DNA by methods familiar to one educated in the art. The mixture is purified to remove contaminating components and aRNA is generated using RNA polymerase and other required components in a suitable buffer. This can be accomplished using commercially available kits (Ambion and Epicentre). During the aRNA synthesis a fluorescent nucleotide can be directly incorporated into the aRNA. The aRNA is then fragmented by standard methods and is applied to a microarray in a buffer suitable to promote the hybridization of the labeled, fragmented aRNA to the probes on the microarray. The excess unbound aRNA is washed from the array and the label detected on a suitable fluoresence detection scanner. Alternatively a capture sequence can be included with the promoter on the oligonucleotide that is ligated to the cDNA and a microarray experiment similar to that described in embodiment 4 performed.

In yet another embodiment of the present invention, a random primed cDNA produced from a bacterial total RNA sample is ligated to a capture sequence at the 3 prime end using RNA ligase and no bridging oligonucleotide. The ligated cDNA/capture sequence mixture is applied to a microarray in a hybridization buffer suitable to allow binding of the cDNA to the probes on the array. The array is washed by methods familiar to one educated in the art. A fluorescent labeled DNA dendrimer with a sequence complementary to the ligated “capture” sequence of the cDNA is added to the array in a second hybridization step and in a suitable hybridization buffer to facilitate binding of the dendrimer to the capture sequence. The excess dendrimer is washed from the array and the label detected on a suitable fluoresence detection scanner.

In another embodiment of the present invention, the 3 prime end of a random primed cDNA produced from a bacterial total RNA sample is ligated to a RNA polymerase promoter using RNA ligase. The random primer used to synthesize the cDNA is designed not to have a phosphate on the 5 prime end and the promoter sequence is designed to have a “blocked” 3 prime end and a 5 prime phosphate. The 3 prime end is blocked during chemical synthesis using a functional group like a primary amine of a carbon linker, like C9 or C18. This configuration of primer and syntheiszed cDNA serves the purposes of fixing the orientation of the promoter on the 3 prime end of the cDNA. Since the primer is located on the 3 prime end of the cDNA, second strand synthesis does not have to be done. The mixture is purified to remove contaminating components and aRNA is generated using RNA polymerase and other required components in a suitable buffer. This can be accomplished using commercially available kits (Ambion and Epicentre). During the aRNA synthesis a fluorescent nucleotide can be directly incorporated into the aRNA. The aRNA is then fragmented by standard methods and is applied to a microarray in a buffer suitable to promote the hybridization of the labeled, fragmented aRNA to the probes on the microarray. The excess unbound aRNA is washed from the array and the label detected on a suitable fluoresence detection scanner. Alternatively, a capture sequence can be included as part of the ligated promoter sequence for detection using a signalling molecule as described above.

In any of the described examples, hybridization of one or more types of targets can be hybridized to the array, e.g., using single or dual channel detection, as known in the art. For each type of target in the assay, a different capture sequence or label is used. The assay can be conducted in a manner in which both hybridization steps are combined into one assay.

Further, more than one type of second oligonucleotide (capture sequence or promoter) can be ligated to the primer such that a polymer of capture sequences or promoters will be coupled to the original primer. Also, the end of the second oligonucleotide can be ligated to either end of the product generated in the initial polymerization reaction with the primer. The second oligonucleotide capture sequence or promoter may be ligated directly (without the need for an initial primer) to the end of the target nucleic acid sequence to be used in the assay.

The foregoing discussion discloses and describes merely exemplary embodiments of the present invention. One skilled in the art will readily recognize from such discussion, and from the accompanying drawings, claims, and the following examples, that various changes, modifications and variations can be made therein without departing from the spirit and scope of the invention as defined in the attached claims.

### EXAMPLE 1

### Expression Analysis using Genisphere Array 350 Kits

Preparation of Tagged cDNA:

In a microfuge tube 2 μg of Total RNA was combined with 4 μg of Random RT Primer. The random prime consisted of a 5 prime extenssion of “GCTTTTT” couple diectly to a sequences containing 9 random nucleotides. Nuclease-free water was added to a final volume of 11 μl. The mixture was heated to 80° C. for 10 minutes to denature the secondary structure of the RNA and cooled on ice for 5 minutes. 4 μl of 5× Superscript H reaction buffer, 2 μl 0.1 M DTT, 1 μl of a dNTP mix containing 10 mM each of dATP, dCTP, dGTP and dTTP, 1 μl Superasin from Ambion Inc. (Austin, Tex.) and 1 μl Superscript II RT enzyme from Invitrogen (Carlsbad, Calif.) were added to the RNA primer mixture on ice. The reaction mixture was incubated at 42° C. for 2 hours. 3.5 μl of 0.5M NaOH/50 mM EDTA was added to stop the reaction and the mixture was, incubated at 65° C. for 10 minutes. The reaction mixture was neutralized by adding 5 μl 1M Tris-HCl pH 7.5 and 21.5 μl TE pH 8.0 to bring sample volume to 50 μl. The sample was purified using QIAquick® PCR Purification Kit, Catalog #28104, from Qiagen (Valencia, Calif.) as directed, except increasing elution spin time to 2 minutes. The final volume was 50 ul after purification. The purified cDNA sample was denatured at 80-90° C. for 10 minutes and transferred to ice immediately for 1-2 minutes.

A ligation reaction was prepared by adding 10 μl of 6× Cy3 or Cy5 Ligation Mix to the purified cDNA. The ligation mix was prepared ahead of time by combining capture sequence oligo and the bridging oligo at a 1 to 2.6 ratio in a ligation buffer such that the ligation buffer components were at 6 times the required final concentration. The final concentration of capture sequences was at 10 picomoles per microLiter. 2.5 μl of T4 DNA Ligase was added. The mixture was mixed gently and incubated at room temperature (15-25° C.) for 1.5 hours. The reacton was stopped by adding 7 μl 0.5M EDTA and incubating at 65° C. for 10 minutes. The sample was purified using QIAquick® PCR Purification Kit, Catalog #28104, from Qiagen (Valencia, Calif.) as directed, except increasing elution spin time to 2 minutes. The final volume was 50 ul after purification. The 50 ul eluted second strand cDNA was concentrated using a Microcon YM-30 Concentrator from Millipore. The final volume of the cDNA was adjusted to 8 ul.

Microarray cDNA Hybridization:

13 ul Nuclease Free water, 25 ul of 2× Formamide Hybridization Buffer (Genisphere Inc, Hatfield, Pa.), and 2 ul of Array 350RP dT Blocker (Genisphere, Inc. Hatfield, Pa.) was added to the 9 ul of cDNA. The cDNA hybridization mixture was heated to 80° C. for 10 minutes and cooled to 50° C. for 10 minutes in an incubater set at 50° C. The microarray was prewarmed to 50° C. or 5 minutes. The cDNA hybridization mixture (50 ul) was pipetted onto the prewarmed array. A coverslip was added and the array was hybridized overnight at 50° C. in a humidified chamber. On the following day the array was washed in a coplin jar in 2×SSC, 0.2% SDS for 15 min at 60-65° C., 2×SSC for 10 min at room temperature and 0.2×SSC for 10 min at room temperature. The array was dried by quickly placing the array in a 50 ml centrifuge tubes and spinning at 1,000 rpm for 2-3 minutes.

3DNA Hybridization

A 3DNA hybridization mixture was set up by mixing 21.5 ul nuclease free water, 2.5 ul Cy3 capture reagent, and 25 ul 2× Formamide-based Hybridization buffer. The mixture was heated to 75-80° C. for 10 minute and then cooled to 50° C. for 10 minutes. The 3DNA hybridization mixture was applied to the array, a coverslip (24×60 mm) was added and the array was hybridized at 50° C. using for 2.5 hours. The array was washed in a coplin jar in 2×SSC, 0.2% SDS for 15 min at 60-65° C., 2×SSC for 10 min at room temperature and 0.2×SSC for 10 min at room temperature. The array was dried by quickly placing the array in a 50 ml centrifuge tubes and spinning at 1,000 rpm for 2-3 minutes. The array was scanned to detect the signal.

### EXAMPLE 2

### Random Prime Amplification

We reverse transcribed 2 ug of human heart total RNA using the Array 350RP primer (Genisphere ligatable random primer) and reverse transcriptase reagents from the Ambion messageAMP aRNA kit in a total volume of 20 ul. The reaction was incubated at 42° C. for 2 hours and stopped by adding 3.5 ul of a 0.5M NaOH/50 mM EDTA solution and heating at 65° C. for 10 minutes. The solution was neutralized by adding 5 ul of 1M Tris-HCl. The volume was adjusted to 50 ul using 1× TE (pH 8.0), and purified using the Qiagen QIAquick PCR Purification kit as directed by the manufacturer. The 50 ul eluted cDNA was heated to 90-95° C. for 10 minutes and cooled for 1-2 minutes on ice before adding 10 ul of ligation mix containing the T7 promoter sequence and ligation buffer. After adding 2.5 ul of T4 DNA ligase, the reaction was incubated at room temperature for 1 hour. The reaction was stopped with 7 ul of 0.5M EDTA and the volume was adjusted to 100 ul with 1× TE. The ligation reaction was purified using the Qiagen QIAquick PCR Purification kit as directed by the manufacturer. The following second strand components from the Ambion messageAMP aRNA kit were added to the 50 ul eluted cDNA as follows: 32 ul nuclease free water, 2 ul second round primers, 10 ul 10× second strand buffer, 4 ul dNTP mix, and 2 ul DNA polymerase. The second strand reaction was incubated at 16° C. for 2 hours, and purified using the Qiagen kit as described above. The 50 ul eluted second strand cDNA was concentrated using a Microcon YM-30 Concentrator from Millipore (Alternatively, ethanol precipitation can be used). The final volume of the cDNA was adjusted to 8 ul. The following in-vitro transcription reagents from the Ambion messageAMP aRNA kit were added to the 8 ul of concentrated cDNA as follows: 2 ul T7 ATP, 2 ul T7 CTP, 2 ul T7 GTP, 2 ul T7 UTP, 2 ul T7 10× Reaction Buffer, and 2 ul T7 Enzyme mix. The IVT reaction was incubated at 37° C. for 6 hours. The template DNA was degraded by adding 2 ul DNase I and incubating the sample at 37° C. for 20 minutes. The DNase I was inactivated by heating to 75° C. for 10 minutes. The aRNA was purified using the Qiagen RNeasy mini kit and following the “Protocol for RNA Cleanup”. The aRNA concentration and quality was analyzed by performing an O.D. reading and running an agarose gel.

The foregoing discussion therefore discloses and describes merely exemplary and preferred embodiments of the present invention. One skilled in the art will readily recognize from such discussion, and from the accompanying drawings, claims, and examples, that various changes, modifications and variations can be made therein without departing from the spirit and scope of the invention as defined in the claims. One skilled in the art may likewise by applying current or future knowledge, adopt the same for use in accordance with the present invention. Yet, having described this invention with regard to specific embodiments, it is to be understood that the description is not meant as a limitation, and that the present application covers all such embodiments, modifications and variations as falling within the scope of the following claims.

1. A method for determining the presence of at least one specific nucleotide sequence in a target nucleic acid extracted from a biological sample, said method comprising the steps of:
preparing a primer oligonucleotide comprising a first bridging sequence with a primer portion composed of a plurality of random nucleotides attached to one end of the first bridging sequence and a terminating end group attached the other end thereof;
hybridizing the plurality of random nucleotides to a complementary portion of one of the at least one specific nucleotide sequence of the target nucleic acid;
initiating reverse transcription from the primer portion of the primer oligonucleotide along the remaining unhybridized portion of the at least one specific nucleotide sequence to yield a sample oligonucleotide comprising a sample sequence complementary to the unhybridized portion of the at least one specific nucleotide sequence;
preparing a capture oligonucleotide comprising a second bridging sequence and a capture sequence;
preparing a bridging oligonucleotide comprising a first complement of the first bridging sequence and a second complement of the second bridging sequence arranged in a manner wherein the hybridization of the bridging oligonucleotide to the first and second bridging sequences of the primer oligonucleotide in the sample oligonucleotide and the capture oligonucleotide, respectively, yields a ligated capture oligonucleotide/sample oligonucleotide strand;
hybridizing the bridging oligonucleotide to the respective first and second bridging sequences of the sample and the capture oligonucleotides in the presence of a ligase reagent to yield the ligated capture oligonucleotide/sample oligonucleotide strand having the capture sequence at the 5′ end of the strand and the sample sequence at the 3′ end of the strand;
contacting the ligated capture oligonucleotide/sample oligonucleotide strand to a microarray having thereon a plurality of features, each of said plurality of features including a probe nucleotide sequence in the presence of a capture reagent having at least one first arm containing a label capable of emitting a detectable signal and at least one second arm having a nucleotide sequence complementary to the capture sequence of the capture oligonucleotide/sample oligonucleotide strand;
treating the microarray at a temperature and for a time sufficient to induce the sample sequence of the capture oligonucleotide/sample oligonucleotide strand to hybridize with the probe nucleotide sequence complementary thereto on the microarray, and then to induce the capture reagent to hybridize to the capture sequence of the capture oligonucleotide/sample oligonucleotide strand hybridized to the microarray wherein the presence of the latter hybridization results in the emission of the detectable signal from the corresponding feature, and the absence thereof results in no emission of the detectable signal from the corresponding feature, thus generating a detectable hybridization pattern for subsequent analysis.

2. The method of claim 1 wherein the plurality of random nucleotides comprises at least two nucleotides.

3. The method of claim 2 wherein the plurality of random nucleotides comprises at least nine nucleotides.

4. The method of claim 1 wherein terminating end group is a phosphate group.

5. The method of claim 1 wherein the first and second bridging sequences each comprise at least two nucleotides.

6. The method of claim 4 wherein the first and second bridging sequences each comprise at least seven nucleotides.

7. The method of claim wherein the capture reagent is selected from the group consisting of dendrimers, carbohydrates, proteins, and nucleic acids.

8. The method of claim 7 wherein the capture reagent is a dendrimer.

9. The method of claim 1 wherein the target nucleic acid is cDNA.

10. The method of claim 1 wherein the ligase reagent is selected from the group consisting of DNA ligase, RNA ligase and mixtures thereof.

11. A method comprising the steps of:
providing a first molecule, said first molecule comprising a cDNA nucleic acid sequence, said first molecule further comprising a primer;
providing a second molecule, said second molecule comprising a nucleic acid sequence, said nucleic acid sequence being a capture sequence for hybridization to a label molecule;
providing a third molecule, said third molecule being an oligonucleotide, said oligonucleotide comprising a first nucleic acid sequence which is complementary to at least a portion of said nucleic acid sequence of said primer of said first molecule; said oligonucleotide further comprising a second nucleic acid sequence which is complementary to at least a portion of said nucleic acid sequence of said second molecule;
hybridizing said third molecule to both said first molecule and to said second molecule; and,
ligating said first molecule to said second molecule.

12. A method comprising the steps of:
providing a first molecule, said first molecule comprising a cDNA nucleic acid sequence, said first molecule further comprising a primer;
providing a second molecule, said second molecule comprising a nucleic acid sequence, said nucleic acid sequence being a promoter sequence;
providing a third molecule, said third molecule being a bridging oligonucleotide, said oligonucleotide comprising a first nucleic acid sequence which is complementary to at least a portion of said nucleic acid sequence of said primer of said first molecule; said oligonucleotide further comprising a second nucleic acid sequence which is complementary to at least a portion of said nucleic acid sequence of said second molecule;
hybridizing said third molecule to both said first molecule and to said second molecule; and,
ligating said first molecule to said second molecule.

13. A method as claimed in claims 11, wherein said first molecule is randomly primed.

14. A method as claimed in claim 11, wherein said capture sequence is provided for hybridization to a complementary sequence of a dendrimer.

15-64. (canceled)

65. A method as claimed in claim 12, wherein said first molecule is randomly primed.


SEQUENCE LISTING (4 sequences)

SEQ ID NO: 1 - DNA, 16 bp, Artificial
  Primer with phosphorylation of synthesized oligonucleotide after synthesis and random mixture of nucleotides
gctttttnnnnnnnnn

SEQ ID NO: 2 - DNA, 7 bp, Artificial
  Second oligonucleotide for serving as a capture molecule and/or as a promoter
nnnnnnn

SEQ ID NO: 3 - DNA, 17 bp, Artificial
  poly dT primer
ttttttttttttttttt

SEQ ID NO: 4 - DNA, 9 bp, Artificial
  Random primer with 5′ bridging sequence
nnnnnnnnn